(19) United States
(12) Patent Application Publication
(10) Pub. No.: US 2023/0372874 A1
(43) Pub. Date: Nov. 23, 2023

(54) DEVICE FOR SUBSTANCE EXCHANGE AND METHOD FOR THE PRODUCTION THEREOF

(71) Applicant: enmodes GmbH, Aachen (DE)

(72) Inventors: Ralf BORCHARDT, Aachen (DE); Ilse Philine RITTER, Aachen (DE); Jens KADEN, Viersen (DE)

(21) Appl. No.: 18/028,329

(22) PCT Filed: Aug. 19, 2021
(86) PCT No.: PCT/EP2021/073007

§ 371 Date: Mar. 24, 2023

(30) Foreign Application Priority Data
Sep. 25, 2020 (DE) 102020125108.4

(51) Int. Cl. B01D 63/02; A61M 1/16

(52) U.S. Cl. CPC B01D 63/023; A61M 1/1698; B01D 2313/041

(57) ABSTRACT

A device for substance exchange between a first medium, in particular blood, and a second medium, in particular a gas/gas mixture, includes a housing element in which there is arranged a hollow-fiber bundle containing substance-permeable hollow fibers which extend axially between the axial ends of the housing element and at their respective axial end region are potted with a potting compound, at least among one another, and around which the first medium can flow and through which the second medium can flow. Also, a method for producing the device is provided.

The invention relates to a device for substance exchange between a first medium and a second medium, comprising a housing element in which there is arranged a hollow-fiber bundle containing substance-permeable hollow fibers, which extend axially between the axial ends of the housing element and at their respective axial end region are potted with a potting compound, at least among one another, and around which the first medium can flow and through which the second medium can flow, wherein the housing element, in particular also the winding core, at at least one of the axial ends, preferably at both axial ends, is closed by a cover element.

The potting with the potting compound can take place not only among the hollow fibers, but preferably also between the housing element inner wall and the hollow fibers, in particular also between the hollow fibers and a winding core surrounded by the hollow fibers, if such a winding core is present. Such winding cores can be provided, on the one hand, to serve as a carrier element for the hollow fibers that are to be wound up on them, in particular if the hollow fibers are wound around the winding core in the form of at least one mat, and, on the other hand, they also define the fluid line, e.g. if one of the fluids flows is conveyed through at least one channel in the winding core, in particular in order to guide this fluid from the outside into the region around the hollow fibers, or vice versa.

The cover element mentioned can have a media connection which is in fluidic communication with the axially open hollow-fiber ends. It is thus possible to deliver the second medium to the hollow fibers via the cover element or also remove said medium from them. Such a cover element is preferably arranged at both axial ends of the housing element and is connected in a sealed manner to the edge regions of the fiber bundle and/or to the housing element.

Depending on the potting material used, leaks may occur at the edges, e.g. if the potting material is not firmly bonded to the inner wall of the housing, as may be the case with silicone, for example. A seal between the hollow-fiber bundle/hollow-fiber winding, potted at the end region, and the housing and/or cover element can then usually be provided by a sealing ring, which is inserted into a ring-receiving groove in the end face of the housing element or of the cover element.

In previous devices for substance exchange, potting and sealing thus represent two separate measures that are typically carried out one after the other and make production complex. Furthermore, the annular groove can itself have a leak that bridges the sealing ring, and it can also form a dead space region in which a fluid can stagnate.

A housing element is preferably an axially extending tubular element, in particular with a circular free internal cross section, although it can in principle have any desired internal cross-sectional shape. The axial ends are preferably open and surrounded by annular end faces. For its part, the housing element can also have connections in order to introduce the first medium into the housing element and/or to discharge it from the housing element. Such connections can be provided, for example, at the axial end regions of the housing element.

Devices of this kind are generally known in the prior art, for example as oxygenators, in which an exchange of substances takes place between blood as the first medium and a gas or gas mixture as the second medium, in particular in order to enrich blood with oxygen and deplete it of carbon dioxide. The hollow fibers used in this case are semipermeable, i.e. not permeable to blood components, but permeable to gas, in particular to oxygen and carbon dioxide.

Such devices are also known in other fields of use, e.g. as devices for carrying out dialysis.

The invention relates to devices of the type mentioned at the outset, irrespective of their specific use, but preferably to oxygenators.

The invention also relates to a method for production of such devices. The production is also known in principle in the prior art.

In the production method, a hollow-fiber bundle, with substance-permeable hollow fibers extending between the axial ends of the housing element, in particular extending at least substantially axially, is arranged in a housing element of the device that is to be formed. Such a bundle is preferably axially longer than the housing element, so that the axial ends of the hollow-fiber bundle protrude from the housing element, and they preferably protrude on both sides.

Such a hollow-fiber bundle can be formed by winding hollow fibers that are linked with warp threads to form mats, e.g. on a winding core. However, the hollow-fiber bundle can also be created by laying or folding of mats.

If the bundle is wound, it can be inserted into the housing element together with the winding core, which in this case is surrounded by the hollow-fiber bundle, preferably lying at the center of the latter. In particular, in such a case, a fluid flow of one of the fluids, in the preferred use e.g. blood, can be provided through the winding core, in particular for which reason the latter, as has already been mentioned, can have at least one channel.

After the housing element has been closed at least at one of its axial ends, preferably at both ends, respectively with a potting cover or with potting covers, the hollow fibers are potted at least among one another, preferably also to the inner wall of the housing element, optionally also to a winding core, with a potting compound at their axial end region covered by the potting cover. In this case, the potting cover usually has a tube portion which protrudes axially from the top surface and surrounds the axial end region of the hollow fibers which protrude from the housing element. As a result, the potting cover can preferably be configured largely in the shape of a pot. The top surface and tube portion can also form separate elements that are connected for the purpose of potting.

The potting process is usually carried out in a centrifuge, with the potting compound being introduced, during the centrifuging, into the housing element closed with the potting cover, e.g. through connections in the potting cover.

According to the known prior art, the potting compound used is a polyurethane compound, for example, which cures and leads to the hollow fibers bonding to one another and also to the inner wall of the housing element, and to the winding core that is preferably used. After curing, such a polyurethane compound actually no longer has any elasticity that would permit a reversible deformation.

After the potting and the hardening or curing of the potting compound, the potting cover is removed, and the potted hollow fibers are opened at their axial ends, which have been potted with the potting compound or were previously closed, e.g. by cutting or sawing them open in a plane perpendicular to the direction of the axial longitudinal extent of the hollow fibers. The housing element, with the potted and opened hollow fibers located therein, is then closed sealingly with a cover element, as has previously been described.

A cured state is preferably understood to be one where the hardening of the potting compound has been completed and does not progress any further. A hardened state can be one where the potting compound has hardened compared to the previous state during potting but where this hardening in particular does not have to be or is not yet completed, but in particular may also be completed.

The aforementioned features, in particular apart from the polyurethane potting compound, may preferably also appear in the invention.

Against this described background with the problems of the previous type of sealing, it is an object of the invention to make available a device of the type mentioned at the outset and a production method of the type mentioned at the outset, which permit simple and reliable creation of a seal between the end face of the housing element and the cover element, and preferably also between the housing element and the hollow-fiber bundle.

This object is achieved, according to the invention, with a production method in which, simultaneously with the potting of the hollow fibers among one another, an outer sealing ring surrounding the hollow-fiber bundle radially on the outside, particularly by a full 360 degrees in the circumferential direction about the longitudinal axis of the hollow-fiber bundle, is created from the potting compound itself, by the potting compound flowing into an annular gap formed between the end faces of housing element and potting cover. Preferably, this annular gap extends in the circumferential direction over a full 360 degrees in the inner wall of the overall housing formed by the housing element and the potting cover.

The annular gap in the overall housing is thus open in a radially inward direction, so that potting compound can penetrate into this annular gap from the interior of the overall housing.

Provision can preferably be made to create not only an outer sealing ring from the potting compound, but also an inner sealing ring. This is provided in particular in those embodiments in which the hollow-fiber bundle is carried as a winding on a winding core, which remains in the housing. In this case, there is not just one annular gap, in this case an outer annular gap, but also an inner annular gap. In the case of such wound hollow-fiber bundles, the bundles are preferably ring-shaped in cross section perpendicular to the longitudinal extent. In particular, this cross-sectional shape then also applies to the potting compound after it has hardened or cured.

In the case of bundles without a winding core, such an embodiment with an inner sealing ring is preferably not provided. In such an embodiment, the hollow-fiber bundle is preferably cylindrical in cross section perpendicular to the longitudinal extent, more preferably with the cross section being completely filled with hollow fibers.

In the method, provision can preferably be made that, simultaneously with the potting of the hollow fibers among one another at the inner circumference of the hollow-fiber bundle, an inner sealing ring is created from the potting compound, by the potting compound flowing into an annular gap, preferably then designated as inner annular gap, formed between the end faces of a winding core, which carries the hollow-fiber bundle, and a cover element in the potting cover.

The outer and/or inner sealing ring is formed directly from the potting compound after the latter has been potted and hardened or cured.

In all possible embodiments, the housing element and the potting cover are preferably adjacent each other at a parting plane, and they contact each other in the parting plane. If the winding core is provided, the same can also apply to this core and to a plug in the cover element.

In one possible embodiment, provision can be made that at least the outer annular gap, preferably the outer and the inner annular gap, adjoins the parting plane in the axial direction, i.e. is arranged in particular in only one of the two elements and, in the direction of the other element, reaches axially to the parting line.

In another possible embodiment, at least the outer annular gap, preferably also the inner annular gap, can lie around the parting plane in the axial direction; in particular, the outer annular gap is then partially arranged in the housing element and in the potting cover, and the inner annular gap arranged partially in the winding core and the plug.

A device of the type mentioned at the outset can thus be produced in which, according to the invention, at at least one of the axial ends of the housing element, preferably at both ends, an outer sealing ring surrounding the hollow-fiber bundle is formed from the potting compound arranged between the hollow fibers, which outer sealing ring extends in the radial direction at least in part between the axial end faces of the housing element and of the cover element and is compressed between these.

In the preferred refinement, at at least one of the axial ends of the winding core which carries the hollow fibers, preferably at both ends, an inner sealing ring arranged on the inner circumference of the hollow-fiber bundle can be formed from the potting compound arranged between the hollow fibers, which inner sealing ring extends in the radial direction at least in part between the axial end faces of the winding core and a plug in the cover element and is compressed between these.

As has already been described for the potting cover, the cover element can preferably be of a largely pot-shaped design, for which purpose the cover element has a top surface and a tube portion extending axially away from the latter and in one piece therewith. The top surface and the tube portion can also form separate elements which are connected for the purpose of forming the whole device housing. The tube portion can surround an axial end region of the potted hollow fibers. The top surface lies axially adjacent to this end region of the hollow fibers, in particular at a distance.

The regional extent is preferably to be understood as meaning that the outer sealing ring, viewed in the radial direction, extends radially outward starting from the radial inner edge of the end faces, but does not completely cover the respective end face in the radial direction. Preferably, the outer sealing ring in the finished device is not accessible from the outside, but is enclosed between the end faces of both elements. However, an embodiment can also be provided in which the sealing ring completely covers the end faces of both elements or even protrudes beyond them in the radial direction.

An end face of the housing element and/or of the cover element and/or potting cover and/or winding core and/or plug or cover element is preferably to be understood as meaning the respective end face, in particular the annular surface, at the axial end region of the element in question, which at least in some areas fulfils a sealing purpose between the mutually opposing elements. Such sealing end faces thus lie axially opposite each other, and in particular they contact each other directly at least in some areas and indirectly in some areas via the respective sealing ring. Other elements/surface elements can moreover be arranged at the axial end of an element, e.g. for the purpose of fastening, e.g. by screwing, latching, etc.

After the potting process, such an outer sealing ring according to the invention is preferably formed by those regions of the potting compound which lie radially outside an (imaginary) envelope surrounding the hollow-fiber bundle, in particular formed at least by those regions which are arranged radially outside the inner wall of the housing element and/or cover element and/or potting cover.

In the case of a preferably provided inner sealing ring, the latter is formed, after the potting process, by those regions of the potting compound which lie radially inside an (imaginary) envelope abutting the inner circumference of the hollow-fiber bundle, in particular formed at least by those regions which are arranged radially to the inside of the outer wall of the winding core and/or plug and/or cover element of a potting cover.

This embodiment according to the invention already has the advantage that the potting compound penetrates in a completely space-filling manner into at least the outer annular gap, preferably also into the inner annular gap, and therefore a respective annular region in the housing element or winding core, which accommodates the sealing ring, is filled with the sealing ring in a manner free from cavities. Stagnation regions can already be prevented in this way.

Provision can preferably be made that the potting compound is formed by a liquid or pasty, in particular at least free-flowing, applicable hardening elastomer with a hardness, in particular in the hardened or cured state, of less than Shore A 100, preferably less than Shore A 60, more preferably less than Shore A 30.

This permits a reversible elasticity of the outer and/or inner sealing ring that is formed, as is known from conventional sealing rings. In addition to the potting of the hollow fibers among one another, the potting compound can thus also provide the properties required for particularly effective sealing, in particular in contrast to conventional polyurethane.

Provision is particularly preferably made to use a silicone/silicone rubber as potting compound, in particular one that can be applied in liquid form in a centrifuge and then hardens/cures, e.g. through crosslinking. It is also possible to use latex as the potting compound.

The outer annular gap to be filled with the potting compound is preferably defined by an axially and radially inwardly open recess in the end face of the housing element and/or of the potting cover. A recess can therefore be provided on the end face of just one of the two elements or of both. The preferably provided inner annular gap to be filled is defined, for example, by an axially and radially outwardly open recess in the end face of the winding core and/or of the cover element in the potting cover. Thus, in the case of the inner annular gap too, a recess can be provided on the end face of just one of the two elements or of both.

Depending on the shape of the annular gap between housing element and potting cover or between winding core and cover element, the respective sealing ring can have different geometries, and therefore different sealing options can be implemented.

At least at the end of the potting and hardening or curing of the potting compound, the outer and/or inner sealing ring formed is integrally or cohesively connected to the potting compound in the regions between the axial ends of the potted hollow fibers. It also preferably remains so in the finished device for substance exchange, although this is not absolutely necessary.

Thus, in one possible embodiment of the method, provision can be made that, with the opening of the hollow-fiber ends, in particular by cutting off bonded hollow-fiber ends or also by separating hollow-fiber ends closed by the manufacturer, the outer sealing ring and/or the inner sealing ring is also separated from the bonded hollow-fiber bundle.

This can be done, for example, if the sealing ring is created in a configuration in which, after the potting cover and/or cover element has been removed, the sealing ring has a region, directed back axially from the annular gap opening in the direction of the housing element, on its radially outer end, and/or a region, directed back axially in the direction of the winding core, on its radially inner end, and lies with this region in a recess which is open at least in the axial direction, e.g. an annular groove, in the end face of the housing element and/or winding core. At the time of the potting process, this annular groove in the housing element or winding core represents an undercut region of the respective annular gap. The separation of the closed hollow-fiber ends can be effected, for example, flush with the end face of the housing element and/or winding core, as a result of which the formed outer and/or inner sealing ring remains in the recess/annular groove, filling the cavity, and is separated from the hollow-fiber bundle.

By placement of the cover element and/or plug, the outer and/or inner sealing ring formed in this way can be compressed between the end faces of cover element and housing element or plug and winding core, e.g. by means of an annular projection pointing from the cover element to the housing element or from the plug to the winding core, which projection is positioned opposite the recess/annular groove. In particular, the plug, preferably when separate from the cover element, can be attached to the winding core, e.g. by latching or screwing or the like.

By contrast, in a preferred embodiment, provision is made that, after the opening of the hollow fiber ends, in particular by cutting off potted hollow-fiber ends or also by separating hollow-fiber ends closed by the manufacturer, the outer sealing ring and/or the inner sealing ring remains attached to the hollow-fiber bundle by cohesive bonding/integral connection, and, by placement of the cover element, the outer sealing ring is compressed between the end faces of cover element and housing element, and/or the inner sealing ring, by placement of the plug, is compressed between the end faces of plug and winding core.

In particular, for this purpose, provision is made to separate the closed hollow-fiber ends in a plane which is spaced apart, in particular by at least by the desired thickness of the sealing ring, from the distal end face plane, i.e. in particular to the most axially outer end face plane of the housing element and/or winding core.

Preferably, the parting plane is spaced apart from the distal end face plane of the housing member and/or winding core by an amount at least 20% greater than the desired axial thickness of the sealing ring formed, more preferably at least 40% greater than the desired axial thickness of the sealing ring formed, particularly at its radially inner region.

This embodiment makes it possible for the outer and/or inner sealing ring to be in one piece with/cohesively bonded to the potting compound between the hollow fibers even in the finished device for substance exchange. This permanent cohesive bonded connection to the potted hollow-fiber bundle affords the advantage that, with the sealing of the cover element relative to the housing element by the outer sealing ring, the hollow-fiber bundle is simultaneously sealed radially outwardly relative to the housing element and the cover element. Furthermore, when the inner sealing ring is provided, the hollow-fiber bundle is also simultaneously sealed radially on the inside relative to the cover element when the plug is placed onto the winding core.

The shape of the annular gap defined between the potting cover and the housing element and/or between the cover element in the potting cover and the winding core can be used, for example, to form an outer sealing ring which, after removal of the potting cover, lies in a recess arranged radially on the inside in the end face of the housing element, and/or to form an inner sealing ring which, after removal of the potting cover and of the cover element, lies in a recess arranged radially on the outside in the end face of the winding core.

The outer sealing ring can, for example, lie flush with the distal end face plane of the housing element, but preferably can also protrude in the axial direction beyond the end face region surrounding the recess, in particular the distal end face plane of the housing element. The inner sealing ring can, for example, lie flush with the distal end face plane of the winding core, but preferably can also protrude in the axial direction beyond the end face region lying radially inward from the recess, in particular the distal end face plane of the winding core. Thus, compression of the sealing ring can be achieved in a particularly simple manner by placement of the cover element and/or of the plug, in particular with the compression taking place into the respective recess.

With regard to the outer sealing ring, the cover element can effect the compression, for example, with an axial end face that is configured overall in only one plane, in particular steplessly, or also by means of an axial, preferably annular projection which is arranged on the end face of the cover element, preferably arranged radially on the inside, and which partially engages in the recess. With regard to the inner sealing ring, the plug can effect the compression, for example, with an axial end face that is configured in only one plane, in particular steplessly, or also by means of an axial, preferably annular projection which is arranged on the end face of the plug, preferably arranged radially on the outside, and which partially engages in the recess.

Likewise, the formed outer sealing ring can lie at a predetermined height on a planar surface region of the end face of the housing element, in particular on a planar surface region that is formed without a recess in the radial extent, and, by placement of the cover element, can be compressed in an axially and radially inwardly open recess in the end face of the cover element with a depth less than the predetermined height.

A preferably formed inner sealing ring can lie at a predetermined height on a planar surface region of the end face of the winding core, in particular on a planar surface region that is formed without a recess in the radial extent, and, by placement of the plug, can be compressed in an axially and radially outwardly open recess in the end face of the plug with a depth less than the predetermined height.

In general, with regard to the outer sealing ring in the device, an axial, preferably annular projection can be arranged radially inwardly on one of the axial end faces of housing element or cover element, which projection lies at least in part in the axially open recess in the end face of the opposite element. Furthermore in general, with regard to the inner sealing ring in the device, an axial, preferably annular projection can be arranged radially on the outside on one of the axial end faces of winding core or plug, which projection lies at least in part in the axially open recess in the end face of the opposite element.

Likewise, the end face of the cover element or of the plug can engage with a recess over the protruding sealing ring, this recess preferably having a depth that is smaller than the protrusion, in order to ensure the compression.

In the finished device, with regard to the outer sealing ring, it can thus generally be provided that the axial end face of the housing element and/or of the cover element has, radially on the inside, an axially and radially inwardly open recess, in which the outer sealing ring lies compressed. In the finished device, with regard to the optionally provided inner sealing ring, it can generally be provided that the axial end face of the winding core and/or of the plug has, radially on he outside, an axially and radially outwardly open recess, in which the inner sealing ring lies compressed.

Where recesses and/or projections on the end faces of elements, e.g. of cover element and/or housing element and/or potting cover and/or cover element and/or plug, are explained in the described embodiments, it should preferably be understood that these extend in the circumferential direction over fully 360 degrees about the longitudinal axis of the hollow-fiber bundle, that is to say are ring-shaped in particular.

In a further possible embodiment, provision can be made that the outer sealing ring is extended in the radial direction outward beyond a projection, preferably a radially inner projection on one of the end faces of housing element (preferred) or cover element, and overhangs it and, by a stepped region at the recess of the opposite element, is bent axially into a region which is arranged set back radially outwardly from the projection, preferably also arranged axially behind the projection.

In a possible similar embodiment, provision can be made that the preferably present inner sealing ring is extended in the radial direction inwardly beyond a projection, preferably a radially outer projection on one of the end faces of winding core (preferably) or plug, and overhangs it and, by a stepped region at the recess of the opposite element, is bent axially into a region which is arranged set back radially inwardly from the projection, preferably also arranged axially behind the projection.

In the method, the outer sealing ring can be formed, for example, in such a way that it covers a radially inner axial projection in the end face of the housing element and protrudes outward in the radial direction and, upon placement of the cover element, is compressed by a recess surrounding the projection in the end face of the cover element and bent in the axial direction.

Similarly, the optionally present inner sealing ring can be formed, for example, in such a way that it covers a radially outer axial projection in the end face of the winding core and protrudes inward in the radial direction and, upon placement of the plug, is compressed by a recess surrounding the projection in the end face of the plug and bent in the axial direction.

Preferred embodiments are explained with reference to the figures.

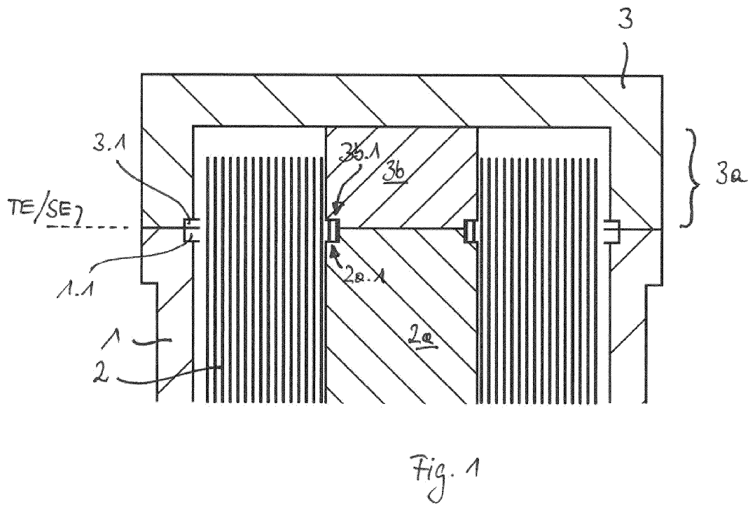

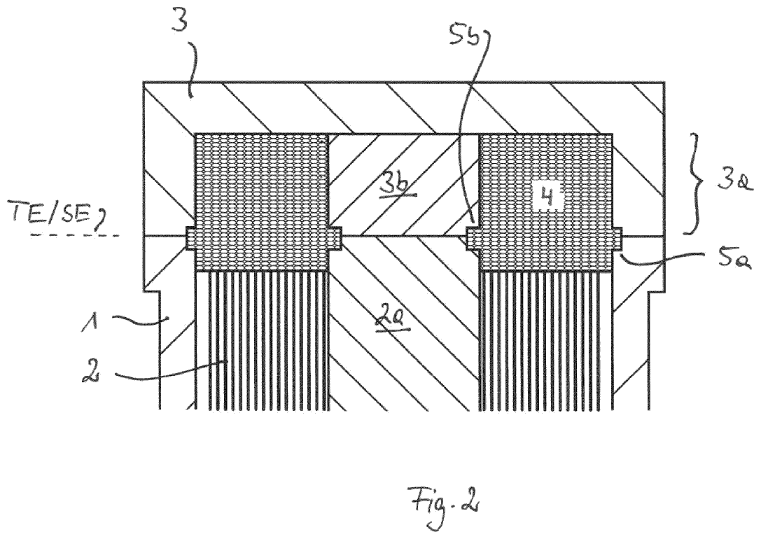

FIG. 1 shows a first step of the production method, in which a hollow-fiber bundle 2 is inserted into the housing element 1, which hollow-fiber bundle 2 protrudes axially beyond the parting plane TE between housing element 1 and potting cover 3 or the distal end face plane SE of the housing element 1. The housing element 1 is closed axially with the potting cover 3, and the end of the hollow-fiber bundle 2 is surrounded by a tubular portion 3a of the potting cover 3.

The hollow-fiber bundle is formed here, for example, by a winding of hollow fibers which are connected to at least one mat with warp threads, for example, with the winding being carried on a winding core 2a, which in particular can also remain subsequently in the housing element 1. A cover element 3b arranged on the potting cover 3 lies axially opposite the winding core 2a, in particular covering its axial end face and being in contact therewith. The cover element 3b can be formed in one piece with the potting cover 3 or is separate from it. For example, in the case of a separate design, it can be clamped between the potting cover 3 and the winding core 2a.

Both annular end faces of housing element 1 and potting cover 3 have respective radially inward recesses 1.1 and 3.1, which are open axially and radially inwardly and thus form an annular gap on the inner wall of the closed arrangement, i.e. of the whole housing. In this embodiment, the annular gap 1.1, 3.1 lies axially around the parting plane TE of the two elements 1, 3, which plane coincides with the distal end face plane SE of the housing element 1. The distal end face of the housing element preferably lies in the same plane as the distal end face of the winding core 2a.

In addition to the outer annular gap thus formed, which results radially on the outside around the hollow fibers and is provided at least in the invention, this embodiment also provides that an inner annular gap also results radially on the inside of the hollow fiber bundle 2. This inner annular gap is formed between the winding core 2a and the cover element 3b. For this purpose, the cover element 3b and the winding core 2a each have respective radially outward recesses 3b.1 and 2a.1, which are open axially and radially on the outside. The inner annular gap 2a.1, 3b.1 also lies axially around the parting plane TE.

FIG. 2 shows the same arrangement after potting at the axial end. The potting compound 4 has here covered the hollow-fiber ends, having possibly closed them if they were not closed by the manufacturer, and has also penetrated into the outer annular gap formed by the recesses 1.1 and 3.1 and also into the inner annular gap formed by the recesses 3b.1 and 2a.1 and has thus formed an outer sealing ring 5a, which surrounds the hollow-fiber bundle 2 on the outside, and formed an inner sealing ring 5b, which is arranged on the inside along the inner circumference of the hollow-fiber bundle 2.

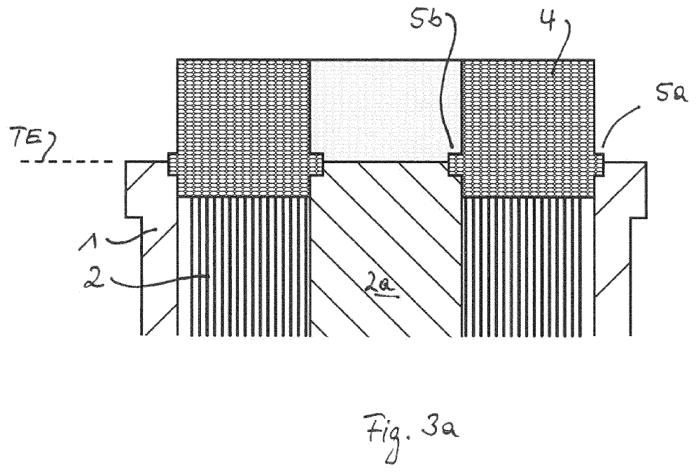

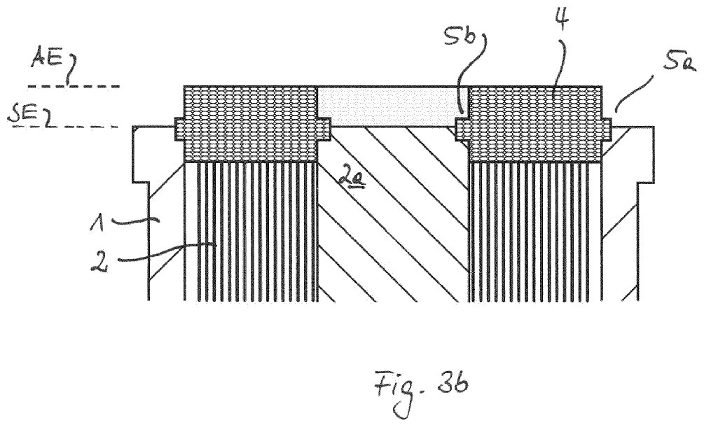

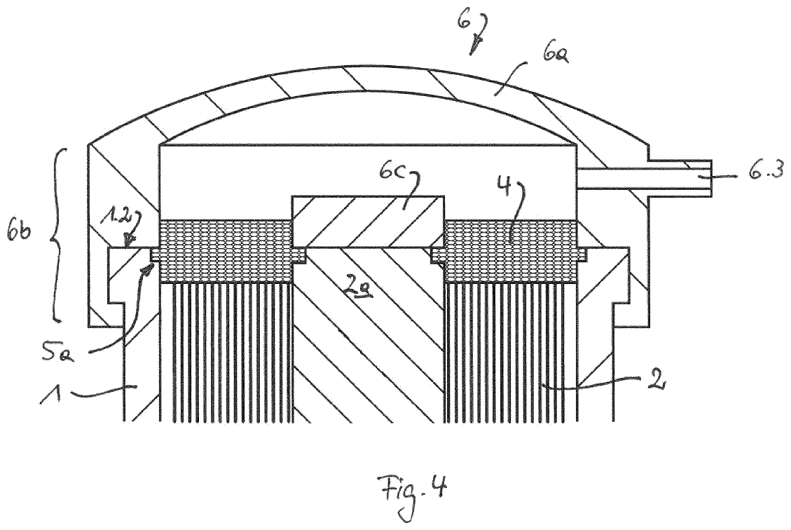

FIG. 3a shows the arrangement after removal of the potting cover 3 and also of the cover element 3b. FIG. 3b shows the same arrangement after some of the potted hollow fibers have been separated in order to open them.

The separation takes place in the separation plane AE at a distance from the sealing rings 5a/5b which, in this embodiment, extends beyond the distal end face plane SE of the housing element 1.

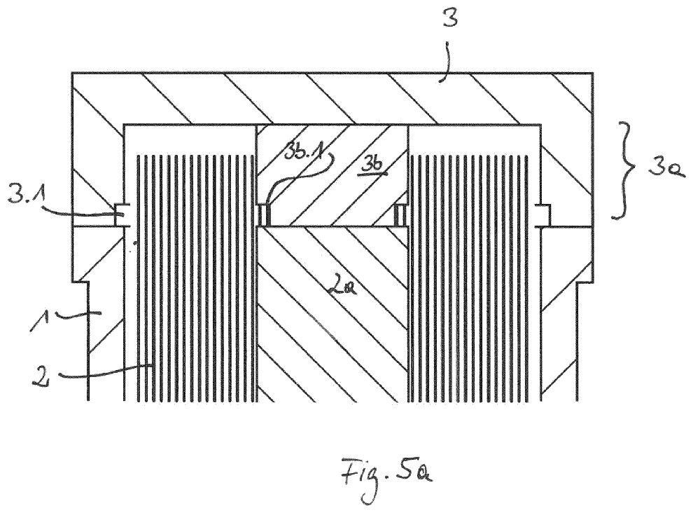

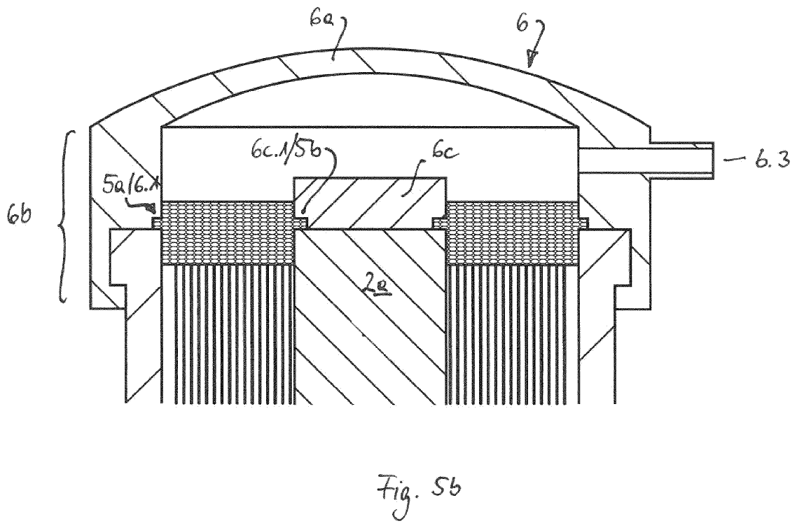

FIG. 4 shows the situation after the cover element 6 of the device has been put in place. The cover element 6 is, in particular similarly to the potting cover 3, pot-shaped and has a top surface, preferably an outwardly curved top surface 6a, from which a tubular portion 6b extends axially in the direction of the housing element 1, which tubular portion 6b in part surrounds the potted hollow-fiber ends.

The cover element 6 also has a connection 6.3 in order to convey gas to the open hollow fibers and away from them. Here, the cover element 6 has no recess in the end face region interacting with the distal end face 1.2 of the housing element 1 for the purpose of sealing, and it compresses the outer sealing ring 5a when the end face is placed onto the axially projecting region of the outer sealing ring 5a.

The inner sealing ring 5b is compressed by a plug 6c, which in particular can be an integral part of the cover element 6 or can also be designed separately from the latter. The plug 6c lies in contact with the winding core 2a. In particular, the plug 6c, preferably when separate from the cover element 6, can be attached to the winding core 2a, e.g. by latching or screwing or the like. This may also apply in all possible embodiments, including those not shown.

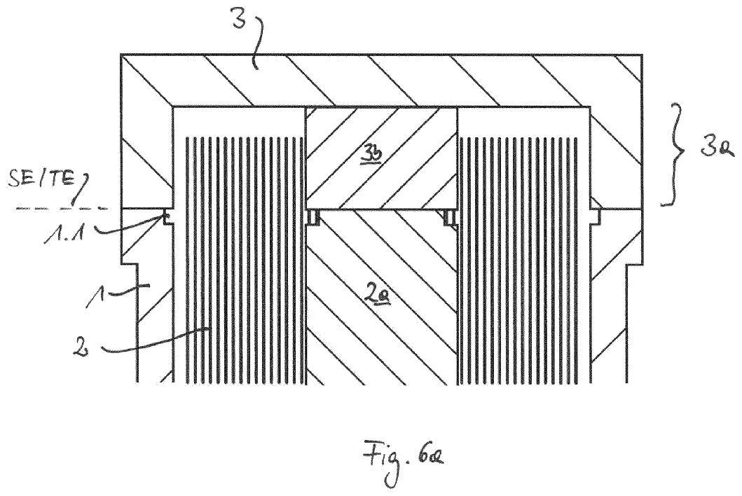

FIG. 5a shows an alternative in which the end face of the housing element 1 lies in a single plane, i.e. has no recess. The equally steplessly planar end face of the winding core 2a lies in the same plane and also has no recess. An axially and radially inwardly open recess 3.1 is arranged only in the end face of the potting cover 3 in order to form the outer annular groove, and an axially and radially outwardly open recess 3b.1 is arranged only in the cover element 3b in order to form the inner annular groove.

In the finished device according to FIG. 5b, the outer sealing ring 5a lies only in a radially inwardly and axially open recess 6.1 and is compressed by the latter, since the depth of the recess 6.1 is less than the height of the sealing ring 5a above the end face of the housing element 1. The inner sealing ring 5b lies only in a radially outwardly and axially open recess 6c.1 and is compressed by the latter, since the depth of the recess 6c.1 in the plug 6c is less than the height of the sealing ring 5b above the end face of the winding core 2a.

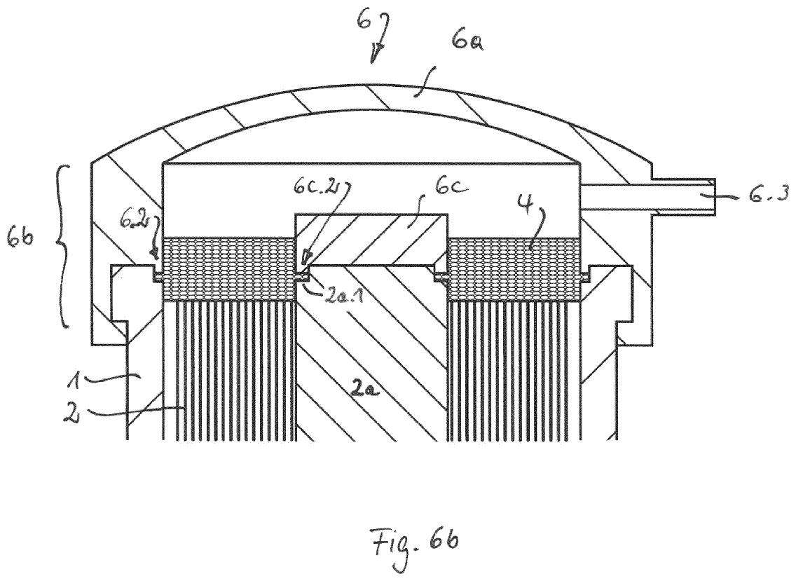

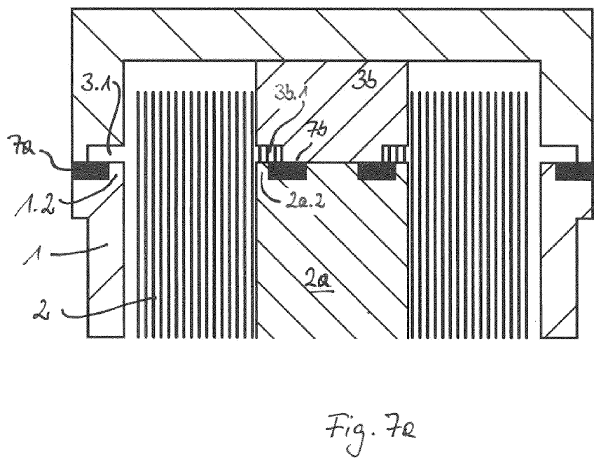

FIG. 6a shows an embodiment in which only one radially inwardly and axially open recess 1.1 is arranged in the housing element 1. The axial end face of the potting cover 3 lies in a single plane (without a recess) and covers the recess 1.1 in the end face of the housing element 1, which is stepped by the recess 1.1.

The outer sealing ring 5a formed according to FIG. 6b thus lies in the distal end face plane SE of the housing element 1 and does not protrude beyond the recess 1.1. For compression, the cover element 6 now has, radially inwardly on its end face, an axial annular projection 6.2 which is directed toward the housing element 1 and which partially engages in the recess 1.1, filled with the sealing ring 5a, on the end face of the housing element 1.

As regards the inner sealing ring 5b, this too lies in the distal end face plane SE of the housing element 1 or of the winding core 2b and does not protrude beyond the recess 2b.1. For compression, the plug 6c now has, radially outwardly on its end face, an axial annular projection 6c.2 which is directed toward the winding core 2a and which partially engages in the recess 2a.1, filled with the sealing ring 5b, on the end face of the winding core 2a.

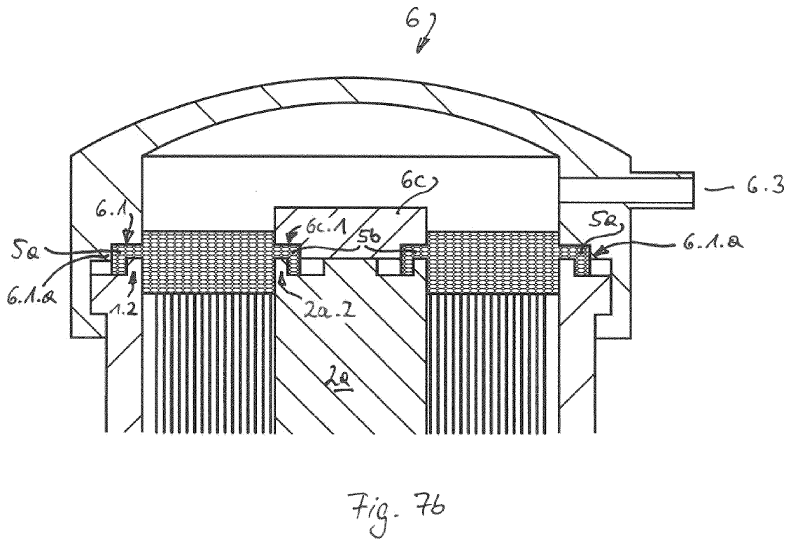

FIGS. 7a and 7b show an embodiment in which the finished outer sealing ring 5a in the finished device (FIG. 7b) covers a radially inner projection 1.2 in the housing element 1 and also projects radially beyond it in a radially outward direction. The protruding part of the sealing ring 5a is bent and compressed in the axial direction by a step 6.1.a of the axially and radially inwardly open recess 6.1 in the end face of the cover element 6. The step 6.1.a delimits the recess 6.1 radially on the outside.

It is also the case that the inner sealing ring 5b in the finished device (FIG. 7b) covers a radially outer projection 2a.2 in the winding core 2a and also protrudes radially beyond it in a radially inward direction. The protruding part of the sealing ring 5b is bent and compressed in the axial direction by a step of the axially and radially outwardly open recess 6c.1 in the end face of the plug 6c. The step delimits the recess 6c.1 radially on the inside.

In FIG. 7a it can be seen that an outer sealing ring 5a overhanging the projection 1.2 can be produced during potting by filling the radially outwardly recessed region radially behind the projection 1.2, for example with a filling ring 7a, which is removed again before the cover element 6 is put in place. The filling ring 7a results in a radially outer region of the annular gap 3.1 that is supported in the axial direction, such that an outer sealing ring 5a extending radially in a straight line can be produced, which protrudes outward beyond the projection 1.2 in the radial direction. In the region filled by the filling ring 7a during the potting process, the outer sealing ring 5a can be bent by the cover element 6 after the filling ring 7a has been removed.

Furthermore, an inner sealing ring 5b overhanging the projection 2a.2 can be produced during potting by filling the radially inwardly recessed region behind the projection 2a.2, e.g. with a filling ring 7b, which is removed again before the plug 6c is put in place. The filling ring 7b results in a radially inner region of the annular gap 3b.1 that is supported in the axial direction, such that an inner sealing ring 5b extending radially in a straight line can be produced, which protrudes inward beyond the projection 2a.2 in the radial direction. After removing the filling ring 7b, the inner sealing ring 5b can be bent by the plug 6c in the region filled by the filling ring 7b during the potting process.

1. A device for substance exchange between a first medium and a second medium comprising a housing element in which there is arranged a hollow-fiber bundle containing substance-permeable hollow fibers which extend axially between axial ends of the housing element and at their respective axial end region are potted with a potting compound, at least among one another, and around which the first medium can flow and through which the second medium can flow, wherein the housing element, at at least one of the axial ends is closed by a cover element which has a media connection in fluidic communication with the axially open ends of the hollow fibers, wherein at at least one of the axial ends of the housing element an outer sealing ring surrounding the hollow-fiber bundle is formed from the potting compound arranged between the hollow fibers, which outer sealing ring extends in the radial direction at least in part between axial end faces of the housing element and of the cover element and is compressed between these, and at at least one of axial ends of a winding core which carries the hollow fibers an inner sealing ring arranged on an inner circumference of the hollow-fiber bundle is formed from the potting compound arranged between the hollow fibers, which inner sealing ring extends in the radial direction at least in part between axial end faces of the winding core and of a plug in the cover element and is compressed between these.

2. The device as claimed in claim 1, wherein the outer and/or inner sealing ring is in one piece with or cohesively bonded to the potting compound between the hollow fibers.

3. The device as claimed in claim 1, wherein the potting compound is formed by an elastomer.

4. The device as claimed in claim 1, wherein the axial end face of the housing element and/or of the cover element has, radially inwardly, an axially and radially inwardly open recess, in which the outer sealing ring lies compressed, and/or the axial end face of the winding core and/or of the plug in the cover element has, radially outwardly, an axially and radially outwardly open recess, in which the inner sealing ring lies compressed.

5. The device as claimed in claim 4, wherein an axial, annular projection is arranged radially inwardly on one of the end faces of housing element or cover element and lies at least in part in the axially open recess in the end face of the opposite element, or an axial, annular projection is arranged radially outwardly on one of the end faces of the winding core or of the plug in the cover element, which projection lies at least in part in the axially open recess in the end face of the opposite element.

6. The device as claimed in claim 5, wherein the outer sealing ring is extended in a radial direction outward beyond the projection and overhangs it and, by a stepped region at the recess of the opposite element, is bent axially into a region which is arranged set back radially outwardly from the projection and axially behind the projection, and/or the inner sealing ring is extended in the radial direction inwardly beyond the projection and overhangs it and, by a stepped region at the recess of the opposite element is bent axially into a region which is arranged set back radially inwardly from the projection and axially behind the projection.

7. A method for production of a device for substance exchange between a first medium and a second medium as claimed in claim 1, wherein a hollow-fiber bundle surrounding a winding core, with substance-permeable hollow fibers extending between axial ends of the housing element, is arranged in a housing element of the device, which hollow fibers, after closure of the housing element at at least one of its axial ends are potted at least among one another at their axial end region which is covered by a potting and, after the potting cover is removed, the potted hollow fibers are opened at their axial ends, and the housing element is tightly sealed with a cover element, wherein, simultaneously with the potting of the hollow fibers among one another, a sealing ring surrounding the hollow-fiber bundle radially on the outside is created from the potting compound, by the potting compound flowing into an annular gap formed between the end faces of housing element and potting cover and, simultaneously with the potting of the hollow fibers among one another at the inner circumference of the hollow-fiber bundle, an inner sealing ring is created from the potting compound, by the potting compound flowing into an annular gap formed between the end faces of a winding core, which carries the hollow-fiber bundle, and a cover element in the potting cover covering the winding core.

8. The method as claimed in claim 7, wherein the annular gap is defined by an axially and radially inwardly open recess in the end face of the housing element and/or of the potting cover, and/or the annular gap is defined by an axially and radially outwardly open recess in the end face of the winding core and/or of the cover element in the potting cover.

9. The method as claimed in claim 7, by cutting off potted hollow-fiber ends or also by separating hollow-fiber ends closed by the manufacturer, the outer sealing ring and/or the inner sealing ring is also separated from the bonded hollow-fiber bundle, wherein the outer sealing ring remains in a recess, open at least in the axial direction, in the end face of the housing element, and, by placement of the cover element, is compressed between the end faces of cover element and housing element, and/or the inner sealing ring remains in a recess, open at least in the axial direction, in the end face of the winding core, and, by placement of the plug, is compressed between the end faces of plug and winding core.

10. The method as claimed in claim 7, after the opening of the hollow-fiber ends by cutting off potted hollow fiber ends or also by separating hollow-fiber ends closed by the manufacturer, the outer sealing ring and/or the inner sealing ring remain attached to the hollow-fiber bundle by cohesive bonding or integral connection, wherein the outer sealing ring, by placement of the cover element, is compressed between the end faces of cover element and housing element, and/or the inner sealing ring, by placement of the plug, is compressed between the end faces of plug and winding core.

11. The method as claimed in claim 7, wherein the shape of the annular gap defined between potting cover and housing element forms an outer sealing ring which, after removal of the potting cover,
a. lies in a recess arranged radially inwardly in the end face of the housing element, protruding in the axial direction beyond the end face region of the housing element surrounding the recess, and, by placement of the cover element is compressed into the recess by an axial projection arranged radially inwardly on the end face of the cover element, or
b. rests at a predetermined height on a planar surface region of the end face of the housing element that is formed in the radial extent without a recess and, by placement of the cover element, is compressed in an axially and radially inwardly open recess in the end face of the cover element, to a depth less than the predetermined height, or
c. covers a radially inner axial projection in the end face of the housing element and protrudes outward in the radial direction and, upon placement of the cover element, is compressed by a recess surrounding the projection in the end face of the cover element and bent in the axial direction.

12. The method as claimed in claim 7, wherein the shape of the annular gap defined between the cover element in the potting cover and the winding core forms an inner sealing ring which, after removal of the cover element and of the potting cover,
a. lies in a recess arranged radially outwardly in the end face of the winding core, protruding in the axial direction beyond the end face region of the winding core lying radially inward from the recess, and, by placement of the plug, is compressed into the recess, in particular by an axial projection arranged radially outwardly on the end face of the plug, or
b. rests at a predetermined height on a planar surface region of the end face of the winding core that is formed in the radial extent without a recess and, by placement of the plug, is compressed in an axially and radially outwardly open recess in the end face of the plug, to a depth less than the predetermined height, or
c. covers a radially outer axial projection in the end face of the winding core and protrudes inward in the radial direction and, upon placement of the plug, is compressed by a recess surrounding the projection in the end face of the plug and bent in the axial direction.
